(19) United States
(12) Patent Application Publication
(10) Pub. No.: US 2018/0344516 A1
(43) Pub. Date: Dec. 6, 2018

(54) MEIBOMIAN GLAND MANUAL MASSAGE DEVICE PAD AND METHOD OF USE

(71) Applicant: Ashley Ophthalmics, LLC, Vicksburg, MS (US)

(72) Inventor: Miles David Ashley, Vicksburg, MS (US)

(73) Assignee: Ashley Ophthalmics, LLC, Vicksburg, MS (US)

(21) Appl. No.: 16/101,818

(22) Filed: Aug. 13, 2018

Related U.S. Application Data
(63) Continuation-in-part of application No. 15/431,738, filed on Feb. 13, 2017.

(51) Int. Cl. A61F 7/02; A61H 7/00; A61F 9/007

(52) U.S. Cl. CPC A61F 7/02; A61F 2007/0004; A61F 9/00772; A61H 7/003

(57) ABSTRACT

A meibomian gland manual device allows for a cost-effective device for use in the home by individuals who suffer from dry eye or for daily eye hygiene. The device allows for a safe self-administration to gently express the meibomian gland, the leading underlying cause of chronic dry eye. The semi-circular projection and an affixed moist heated pad, combined with the action of gently applying pressure to the eyelid while following the natural direction of the eyelid, increases the effectiveness of expressing the meibomian gland. The material of the device allows for ease of cleaning for repeated use. Combining the device with either a warm shower or applying a warm compress to the eyes increases the effectiveness of the device to induce a manual extraction of the gland contents.

### RELATED APPLICATIONS

This application is a continuation-in-part of U.S. Ser. No. 15/431,738 filed on Feb. 13, 2017, hereby incorporated by reference in its entirety. Applicant claims the benefit thereof.

### STATEMENT REGARDING FEDERALLY SPONSORED RESEARCH OR DEVELOPMENT

None

### NAMES OF PARTIES TO A JOINT RESEARCH AGREEMENT

None

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

None

### STATEMENT REGARDING PRIOR DISCLOSURE BY THE INVENTOR OR A JOINT INVENTOR

None

### BACKGROUND OF THE INVENTION

### Field of the Invention

The following apparatus and method generally relates to the treatment of dry eyes and more specifically to the application of a manual massage device to the eyelids to cause the expression of the meibomian glands.

### Description of Related Art Including Information Disclosed Under 37 CFR 1.97 and 1.98

Dry eye is a common multifactorial condition that affects approximately one in four adults. Any individual can develop dry eye, although the most common demographics are the elderly and women. (Gayton, Johnny L., Etiology, Prevalence and Treatment of dry eye disease, Clin. Ophthalmol. Vol. 3 2009, 405-412). Untreated dry eye can cause many impairments to an individual's quality of life. Dry eye is most commonly characterized by burning, itching, and redness of the eyes. In later stages of the condition, an individual can suffer damage to the eyes in the form of scratches to the cornea leading to blurred vision, temporary loss of vision and in rare cases permanent loss of vision. Up to 90 percent of dry eye is caused by or associated with meibomian gland dysfunction. Daily expression of the meibomian gland can alleviate dry eye and prevent damage to the eyes. It can also be beneficial for individuals without the diagnosis of dry eye for daily eye hygiene.

The meibomian gland secretes the necessary oily compound, known as meibum. The meibum, along with the aqueous and mucin layers of the tear film, keeps the eye moist, washes away foreign matter thereby reducing the risk of infection and keeps the eyes smooth and clear, facilitating an individual's ability to see clearly. When the meibomian gland dysfunctions, it can lead to blockages within the glands themselves, causing the oil secretion to become granular and eventually plugged, so that less of the necessary oily compound is produced. Eventually, the glands will become blocked with thickened secretions. Chronically clogged glands will become unable to secrete oil, which results in a permanent change in the tear film and the condition known as chronic dry eye. For most patients, once they develop meibomianitis, the condition lasts a lifetime. (Gayton, 2009). Untreated meibomian gland disease can lead to ocular infection and/or inflammation of the eyelids. If untreated, meibomianitis can lead to corneal disease, which can lead to uncorrectable blurred vision and blindness in severe cases. (Gayton, 2009).

To prevent the loss of tear production in the eyes, it is recommended individuals gently express the glands on a regular basis. (American Optometric Association, 2017). Meibomian gland stagnation or blockage can be relieved in hopes of preventing or relieving dry eye secondary to meibomian gland dysfunction. Hot compresses and lid massages are often used in tandem to express the glands, whereas the hot compress “melts” or substantially decreases the viscosity of the thickened secretions, thereby allowing the expression of the oily compound by massaging the gland. For best results, an individual should massage the gland once to twice daily to prevent the buildup of the thickened secretions.

Prior art devices designed for use on the eyelids include devices that apply heat or electro-mechanical massaging or a combination of the two. The drawbacks of these devices are twofold: one, the cost of the device and two, the complexity and potential difficulty for a layperson to use the device. Previous devices all have either a heating element or electro-mechanical element or a combination of both. Both elements often require a power source, a battery or a need to plug the device in. The complexity of the device increases the cost of materials as well as production of the device. This cost is ultimately passed onto the consumer. Further, the complexity of the prior devices increases the likelihood of a malfunction of one of the elements necessitating the purchase of another device, thereby increasing the cost of maintaining the necessary daily regimen.

The very complexity of the devices in the prior art can act as an impediment for the necessary daily regimen as well. Previous devices have moving parts, are often bulky to accommodate the motor, heating element and power source, creating difficulty for individuals to use. The difficulty of cleaning and caring for the device increases as the complexity of the device increases. The difficulty associated with use and care of the previous devices can impede the individual's use, preventing the necessary daily regimen prescribed. It is the objective of the present invention to provide an affordable and easy to use device. Eliminating the use of heating or electro-mechanical parts decreases the cost of production and assembly of the device. Without those elements the device is smaller, allowing an ease of use not present in the prior art. The streamlined design of the present subject matter allows for individuals to quickly learn how to massage their lids, while also being easy to care for. Combined, this would allow an individual to follow the regimen prescribed by their physician with a comfort and ease not present in the prior art.

### BRIEF SUMMARY OF THE INVENTION

This summary describes several embodiments of the presently disclosed subject matter, and, in many cases, lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

The inventive subject matter of this invention includes: a meibomian gland manual massage device made of: a rigid semi-circular projection. The rigid semi-circular projection has a first face and a second face, wherein the first face has a concave shape configured to contact an edge of an eyelid and a second face; a handle; and a plurality of arms to connect the second face of the rigid semi-circular projection to the handle, wherein the meibomian gland manual massage device further includes a pad affixed to the first face. The pad facilitates adding heat to the treatment by soaking the pad in water prior to use.

The inventive subject matter also includes a method to manually massage the meibomian gland. The steps of this method include: placing a meibomian gland manual massage device made of a rigid semi-circular projection upon an upper or a lower eyelid, flush to an eyelid's eyelashes; the semi-circular projection having a first face and a second face, wherein the first face has a concave shape and has a pad configured to contact an edge of an eyelid, after having soaked the pad in warm water, and a second face opposite the first face; applying pressure to the eyelid with the meibomian gland manual massage device, wherein a user pulls the eyelid through a normal range of vertical motion of the eyelid to express the meibomian gland; and releasing pressure to the eyelid with the meibomian gland manual massage device, wherein the user slowly returns the eyelid to a relaxed position.

Advantages of the presently disclosed subject matter will become evident to those of ordinary skill in the art after a study of the description, Figures, and non-limiting Examples in this document.

### BRIEF DESCRIPTION OF THE DRAWINGS

FIG. 1 illustrates an embodiment of a meibomian gland manual device.

FIG. 2 is a front view for an embodiment of a meibomian gland manual device.

FIG. 3 illustrates the method of placement of the pad on to the meibomian gland manual device.

FIG. 4 illustrates the step of washing the pad affixed to the meibomian gland manual device.

FIG. 5 illustrates the method of placement of the eyelid device on the lower eyelid.

FIG. 6 illustrates the method of movement of the eyelid device on the lower eyelid.

FIG. 7 illustrates the method of placement of the eyelid device on the upper eyelid.

FIG. 8 illustrates the method of movement of the eyelid device on the upper eyelid.

### DESCRIPTION OF EXEMPLARY EMBODIMENTS

The present invention can be understood more readily by reference to the following detailed description of the invention. It is to be understood that this invention is not limited to the specific devices, methods, conditions or parameters described herein, and that the terminology used herein is for describing embodiments by way of example only and is not intended to be limiting of the claimed invention. Also, as used in the specification including the appended claims, the singular forms “a,” “an,” and “the” include the plural, and reference to a numerical value includes at least that value, unless the context clearly dictates otherwise. Ranges can be expressed herein as from “about” or “approximately” one value and/or to “about” or “approximately” another value. When such a range is expressed, another embodiment includes from the one value and/or to the other value. Similarly, when values are expressed as approximations, by use of the antecedent “about,” it will be understood that the value forms another embodiment. All combinations of method or process steps as used herein can be performed in any order, unless otherwise specified or clearly implied to the contrary by the context in which the referenced combination is made.

These and other aspects, features and advantages of the invention will be understood with reference to the detailed description herein and will be realized by means of the various elements and combinations particularly pointed out in the appended claims. It is to be understood that both the foregoing general description and the following detailed description of the invention are exemplary and explanatory of preferred embodiments of the inventions and are not restrictive of the invention as claimed. Unless defined otherwise, all technical and scientific terms used herein have the same meaning as commonly understood by one of ordinary skill in the art to which this invention belongs.

A meibomian gland manual massage device described herein is a practical and cost-effective way to self-administer the treatment for one of the leading causes of dry eye, namely, preventing the blockage of the glands by daily gently expression of the glands. The manual device is a safe and an effective means to express the glands while also being cost effective. The meibomian gland manual massage device is designed with a rigid semi-circular projection and an affixed pad on the projection, off-set from the handle. The meibomian gland manual massage device can be used on the eyelid safely and comfortably by the consumer on their own, without the need for a medical professional to be present. The rigid semi-circular projection and the affixed pad are configured to conform to the edge of the eyelid, flush to the eyelashes for both the upper and lower eyelid. The apparatus is made of a synthetic polymer.

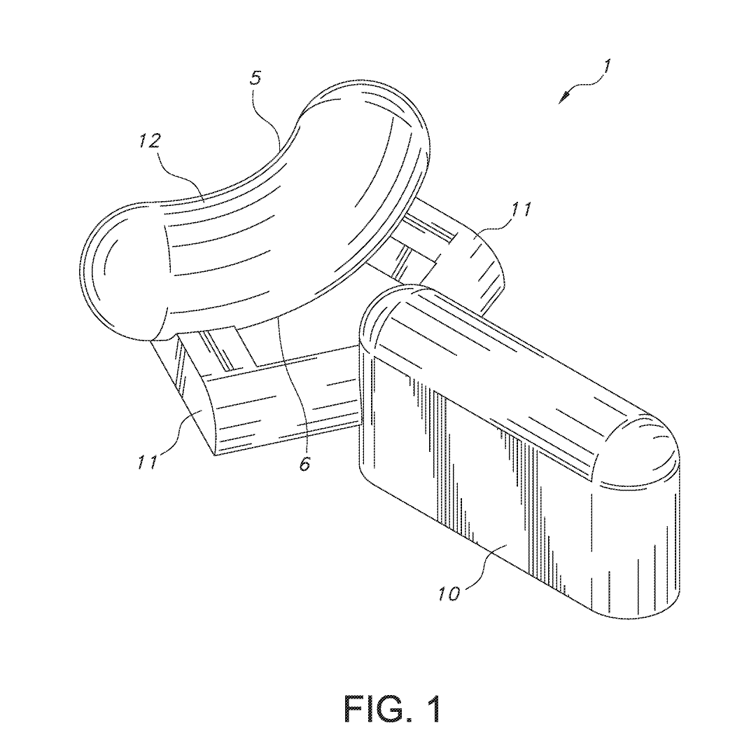

Now referring to FIG. 1, a meibomian gland manual massage device 1 is shown with a handle 10, a plurality of arms 11 and a rigid semi-circular projection 12. The rigid semi-circular projection 12 has a first face 5 and a second face 6. The first face 5 has a concave shape and is configured to contact an edge of an eyelid. The portion of the first face 5 that contacts the eyelid, in one exemplary embodiment, is 16 mm in length. The handle 10, in one exemplary embodiment, is 30 mm from end to end and is off-set from the plurality of arms 11, such that it does not interfere with the movement of the semi-circular projection 12 as it contacts the eyelid.

Upon the application of force, through the handle 10, the rigid semi-circular projection 12, moves the eyelid in an up and down motion, as it conforms to the eyeball. The plurality of arms 11 are connected on both the left and right underside of second face 6 of the semi-circular projection 12. The plurality of arms 11 are connected to the top of the handle 10 along the second face 6. The second face 6, in one exemplary embodiment, is convex in shape. The plurality of arms 11 are spaced apart to allow for the application of equal force to the eyelid.

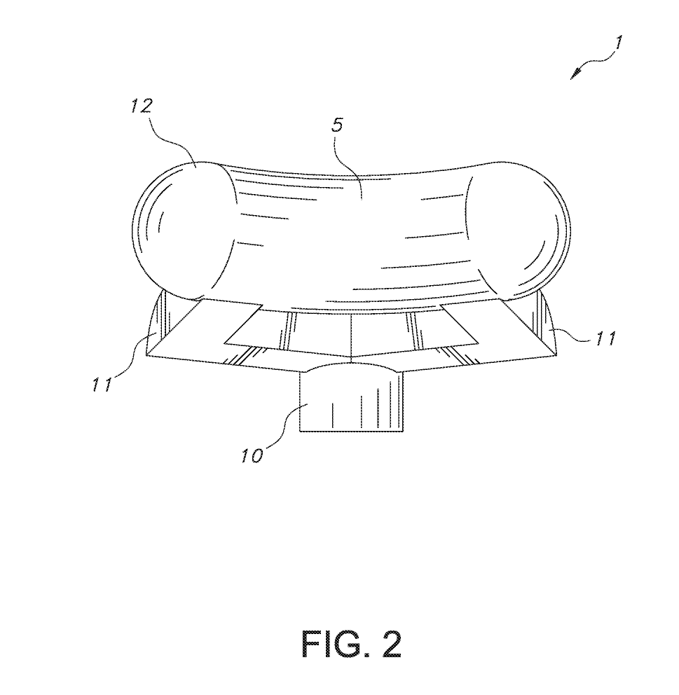

A front view of the meibomian gland manual massage device 1 as seen in FIG. 2, the connections between the semi-circular projection 12 and handle 10 are shown from a different angle. Looking directly on towards the semi-circular projection 12, the first face 5 has a concave shape and it is configured to contact an edge of an eyelid. The plurality of arms 11 connect on both the left and right to the underside of the semi-circular projection 12. The plurality of arms 11 then connect to the top of the handle 10. In one exemplary embodiment, the end of the edge of the handle 10 measures 6.0 mm to the center of second face 6 and 14.0 mm to the center of the first face 5.

The meibomian gland manual massage device 1 can be made from any synthetic polymer that holds the appropriate strength profile and can be produced with injection molding. The meibomian gland manual massage device 1 can be produced using injection molding and is designed to be reusable with a recommended replacement of every three months. Preferably, the device is made of the synthetic polymer nylon. Nylon is an excellent material for the meibomian gland manual massage device 1 because of its strength profile and generally hypoallergenic nature. Nylon's hypoallergenic nature allows a broader spectrum of individuals who can safely use the device for their eyecare. Nylon is also preferred because of the ease of care associated with cleaning the material. The device can be disinfected for further use with simple mild soap, such as hand soap, instead of expensive and possibly caustic chemicals.

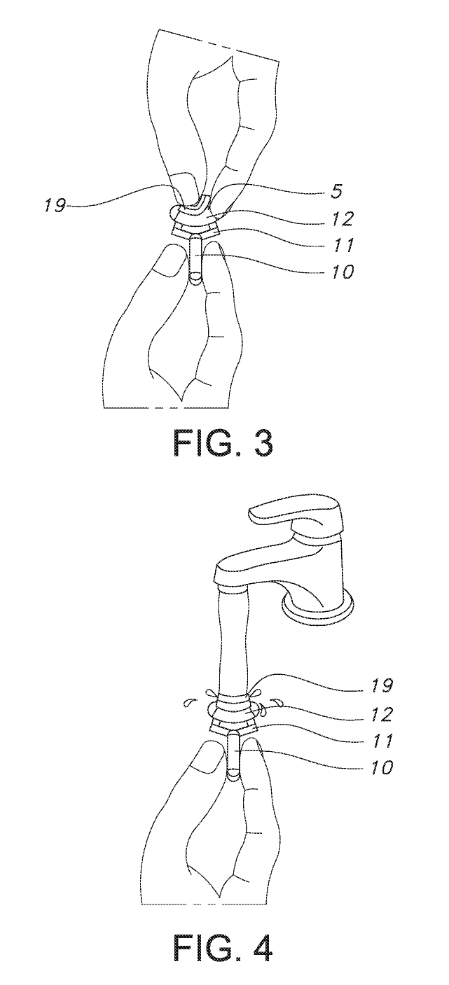

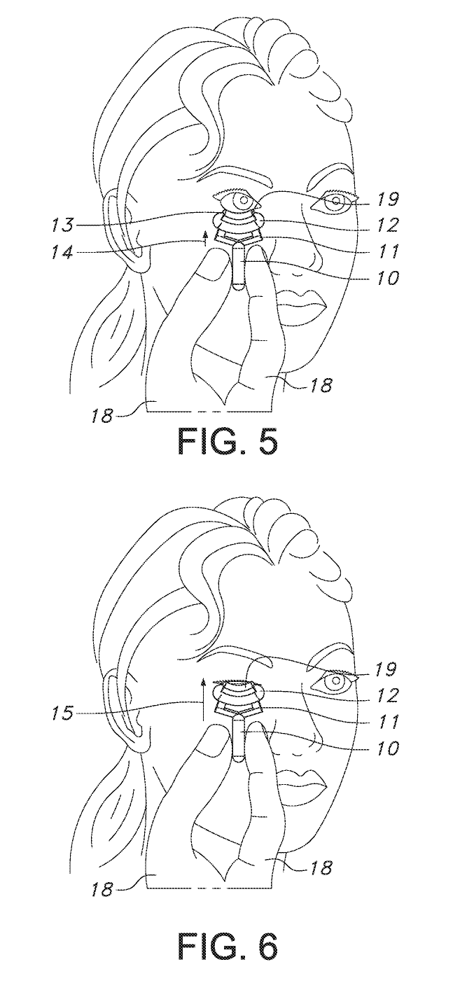

Now referring to FIGS. 3-4 after cleaning the semi-circular projection 12 of the meibomian gland manual massage device 1 with mild soap and water. An adhesive layer (not shown) is removed from a pad 19 and the pad 19 is affixed to the first face 5 of the body of the meibomian gland manual massage device 1. The pad 19 is made of an absorbent material that can be heated and retain its shape such as a polyurethane foam. The pad 19 in one embodiment is 1 inch long, 0.25-inch-wide and 0.125 inch thick. The pad 19 is affixed to the body of the meibomian gland manual massage device 1 along first face 5 and the pad 19 is soaked in warm water for several seconds prior to use as shown in FIGS. 5-8 to provide heat to the effective area.

FIGS. 5-8 illustrate the use of the meibomian gland manual massage device 1. FIG. 5 illustrates how to place the meibomian gland manual massage device 1 to the lower eyelid. While holding the handle 10 between a user's fingers 18, the user places the semi-circular projection 12, having a first face 5 and a second face 6, wherein the pad 19 affixed to first face 5 with a concave shape contacts an edge of an eyelid, on the edge of the lower eyelid, flush to the eyelashes 13. Applying gentle pressure, but not to the point of discomfort or pain, the meibomian gland manual massage device 1 is moved upwards towards the forehead 14. Once the eyelid has been gently pulled to its upward limit with the meibomian gland manual massage device 1, the device can then be used to pull the lid gently downward again into its resting position. This cycle of up and down movement can be repeated several times to ‘milk’ the glands.

FIG. 6, illustrates the movement by maintaining the gentle pressure upwards, relaxing the eyelid and slowly pulling the eyelid upwards through its normal range of vertical motion stretching the eyelid 15, but not to the point of pain or discomfort. Preferably this action is to be repeated five to ten times.

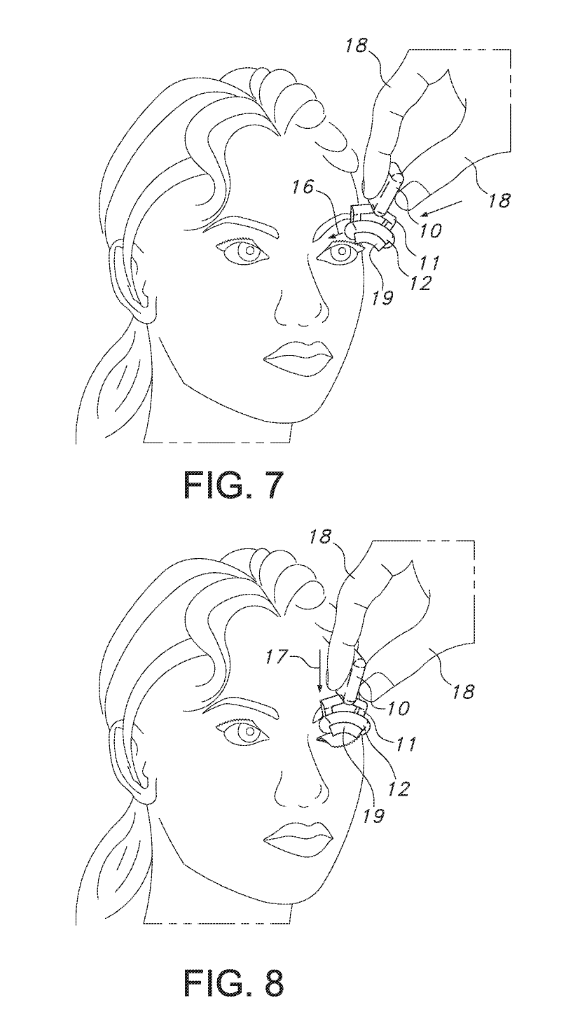

FIG. 7 illustrates how to place the meibomian gland manual massage device 1 to the upper eyelid. Holding the handle perpendicular to the eyeball 10, the user places the semi-circular projection 12 wherein the pad 19 affixed to first face 5 with a concave shape contacts the edge of the upper eyelid, flush to the eyelashes 16. In FIG. 8, the user exerts a gentle pressure downward towards the chin pulling the eyelid down through the normal range of vertical motion of the eyelid 17, gently stretching the eyelid but not to the point of pain or discomfort. Preferably this action is to be repeated five to ten times.

The procedure illustrated in FIGS. 5-8 are to be repeated for the eyelids of the individuals' other eye. The techniques described in FIGS. 5-8 are most effective after the eyelids have been warmed. This can be achieved by using the apparatus and techniques, after a warm shower or after placing a warm compress, such as a warm washcloth, to the closed eyelids for three to five minutes. The warming of the eyelids softens the oils in the meibomian glands allowing them to be expressed more easily using the above described techniques. This cycle of up and down movement can be repeated several times to ‘milk’ the glands. It is recommended the device is cleaned prior to and after use with a mild soap, such as hand soap, and water.

The apparatus and techniques described above allow for long term, regular maintenance of the meibomian gland function by individuals at home. Under previous arrangements, maintenance would involve visits to a medical eyecare professional for expensive meibomian gland drainage procedures. Between those visits, the individual had to rely upon at home maintenance devices which were often expensive, bulky and difficult to use. In contrast, the apparatus and techniques discussed above give an individual a cost-effective option which is easy to use for safe and effective regular maintenance of expressing the meibomian glands.

The foregoing detailed description has been presented for purposes of illustration and description. It is not intended to be exhaustive or to limit the invention to the precise form disclosed. Many modifications and variations are possible considering the above teachings. The described embodiments were chosen to best explain the principles involved and their practical applications, to thereby enable others skilled in the art to best utilize the various embodiments and with various modifications as are suited to the use contemplated. It is intended that the scope of the invention be defined by the claims appended hereto.

1. A meibomian gland manual massage device comprising:
a rigid semi-circular projection, the semi-circular projection having a first face and a second face, wherein the first face has a concave shape configured to contact an edge of an eyelid and a second face opposite the first face;
a handle; and
a plurality of arms to connect the second face to the handle, wherein the meibomian gland manual massage device further comprises a pad affixed to the first face.

2. The meibomian gland manual massage device of claim 1, wherein the manual device is made of a synthetic polymer.

3. The meibomian gland manual massage device of claim 2 wherein the manual massage device is comprised of Nylon.

4. The meibomian gland manual massage device of claim 1, wherein the second face is convex.

5. The meibomian gland manual massage device of claim 1, wherein the pad is made of a polyurethane foam.

6. A method to manually massage a meibomian gland comprising the steps:
contacting the pad of the meibomian gland manual massage device of claim 1 with an edge of an eyelid;
applying pressure to the eyelid with the meibomian gland manual massage device, wherein a user pulls the eyelid through a normal range of vertical motion of the eyelid to express the meibomian gland; and
releasing pressure to the eyelid with the meibomian gland manual massage device, wherein the user slowly returns the eyelid to a relaxed position.

7. The method of claim 6, wherein the user pulls the eyelid through a normal range of vertical motion of the eyelid five to ten times for the lower eyelid.

8. The method of claim 6 wherein the user pulls the eyelid through a normal range of vertical motion of the eyelid five to ten times for the upper eyelid.

9. The method of claim 6, further comprising: warming the eyelid before use of the meibomian gland manual massage device.

10. The method of claim 9, wherein the eyelid is warmed for three to five minutes prior to applying the meibomian gland manual massage device to the eyelid.

11. The method of claim 6, wherein the meibomian gland manual massage device is moved upwards towards the forehead, while maintaining pressure.

12. The method of claim 6, wherein the meibomian gland manual massage device is moved downwards towards the chin, while maintaining pressure.

13. The method of claim 6 further comprising: heating the pad prior to contacting the edge of an eyelid.

14. The method of claim 13 further comprising: heating the pad by contacting the pad with warm water.
